(19) United States
(12) Patent Application Publication
(10) Pub. No.: US 2019/0185812 A1
(43) Pub. Date: Jun. 20, 2019

(54) METHOD OF DIFFERENTIATING NEURAL STEM CELLS OR NEURAL PRECURSOR CELLS INTO DOPAMINE NEURONS

(71) Applicant: Industry-University Cooperation Foundation Hanyang University, Seoul (KR)

(72) Inventors: Chang-Hwan PARK, Seoul (KR); Sang Mi Kim, Andong-si (KR)

(73) Assignee: Industry-University Cooperation Foundation Hanyang University, Seoul (KR)

(21) Appl. No.: 16/220,048

(22) Filed: Dec. 14, 2018

(30) Foreign Application Priority Data
Dec. 15, 2017 (JP) 2017-240499
Dec. 15, 2017 (KR) 10-2017-0172855

(51) Int. Cl. C12N 5/0793; A61K 35/30; C12N 15/85

(52) U.S. Cl. CPC C12N 5/0619; A61K 35/30; C12N 2506/08; C12N 2501/60; C12N 15/85

(57) ABSTRACT

The present invention relates to a method of differentiating neural stem cells or neural precursor cells into dopamine neurons and more particularly, a method of differentiating into dopamine neurons, in which chromosomal stability is maintained by transfecting neural stem cells or neural precursor cells with mRNA of a dopamine neuron-inducing transcription factor under time-based control. The method of differentiating into dopamine neurons, according to the present invention, may enable preparation of mature and functional dopamine neurons having chromosomal stability by synthesizing a dopamine neuron-inducing transcription factor into a mRNA form, which has no risk of genetic modification, and transfecting the synthetic mRNA, unlike existing methods using retroviral vectors, and thus may be usefully used in the clinical field for the treatment of Parkinson's disease.

### CROSS-REFERENCE TO RELATED APPLICATION

This application claims priority to and the benefit of Korean Patent Application No. 10-2017-0172855, filed on Dec. 15, 2017, and Japanese Patent Application No. 2017-240499, filed on Dec. 15, 2017, the disclosure of which is incorporated herein by reference in its entirety.

### BACKGROUND

### 1. Field of the Invention

The present invention relates to a method of differentiating neural stem cells or neural precursor cells into dopamine neurons, and more particularly, to a method of differentiating into dopamine neurons, in which chromosomal stability is maintained by transfecting neural stem cells or neural precursor cells with mRNA of a dopamine neuron-inducing transcription factor under time-based control.

### 2. Discussion of Related Art

Parkinson's disease (PD) is a degenerative brain disorder of the central nervous system that is accompanied by movement disorders such as akinesia, rigidity, and tremors, and is known to be caused by a reduction in the number of dopamine neurons that must reach the striatum, due to progressive death of dopamine neurons located in the substantia nigra of the midbrain.

Currently, there is a method of administering levo-DOPA (L-DOPA), which is a dopamine precursor, as Parkinson's disease therapy, but it causes side effects such as nausea, restlessness, sleep disorders, hypotension, stereotyped movements, hallucinations, delusion, and the like, and other methods also have an effect of temporarily improving symptoms. However, it was observed about 15 years ago that, when the brain tissue of a stillborn fetus containing dopamine neurons was transplanted into a patient with Parkinson's disease, dopamine neurotransmission was restored, thus enabling dopamine neurons to reach the striatum, and accordingly, akinesia symptoms were alleviated in some patients with Parkinson's disease (NeuroRX, October 2004, Volume 1, Issue 4, pp 382-393). As a result, the concept of cell therapy, which is expected to alleviate symptoms by replacing damaged cells with new ones from the outside via transplantation, has been proposed. However, this method also has ethical and technical problems, and thus to address these problems, research on cell therapy using neural stem cells, which are capable of producing the same cells as themselves by cell division and differentiating into different specific cells according to differentiation stimuli, has emerged. As a method of inducing dopamine neurons by using existing neural stem cells, a method using a retrovirus is known. However, this method has problems such as the risk of genetic modification and mutagenesis, and further has limitations such as not being suitable for clinical application. Thus, a RNA-based or protein-based gene delivery method, which is used in studies on induced pluripotent stem cells, iPSCs, has drawn attention as an appropriate method of generating DNA-free dopamine neurons. However, the protein delivery method requires preparation and purification of a large amount of proteins in the expression of a target gene, and the RNA delivery method uses a RNA virus, and thus has a limitation in that an additional selection process is required to maintain a virus-free state.

Therefore, there is a need to develop a method capable of differentiating neural stem cells into stable dopamine neurons through a novel, effective gene expression method.

### SUMMARY OF THE INVENTION

As a result of studying a method for addressing the above-described existing problems and of efficiently and stably differentiating neural stem cells or neural precursor cells into dopamine neurons, the inventors of the present invention found that functional differentiation into dopamine neurons, in which chromosomal stability was maintained, was enabled by transfecting neural stem cells with mRNA of a dopamine neuron-inducing transcription factor, thus completing the present invention based on these findings.

Thus, an object of the present invention is to provide a method of differentiating neural stem cells or neural precursor cells into dopamine neurons and dopamine neurons prepared by the method.

Further, another object of the present invention is to provide a cellular therapeutic agent for treating Parkinson's disease, which includes the above-described dopamine neurons.

Technical problems to be achieved by the present invention are not limited to the aforementioned technical problems, and other unmentioned technical problems will become apparent from the following description to those of ordinary skill in the art.

In order to achieve the object of the present invention as described above, the present invention provides a method of differentiating neural stem cells or neural precursor cells into dopamine neurons, including transfecting neural stem cells or neural precursor cells with mRNA of a dopamine neuron-inducing transcription factor.

In one embodiment of the present invention, the dopamine neuron-inducing transcription factor may be nuclear receptor related 1 (Nurr1) or Forkhead box protein A2 (FoxA2).

In another embodiment of the present invention, the method may further include inducing differentiation of neural stem cells or neural precursor cells into neurons for about 5 days to about 10 days before the transfecting process.

In still another embodiment of the present invention, the mRNA may be repeatedly transfected into neural stem cells or the neural precursor cells at an interval of about 1 day to about 2 days.

In addition, the present invention provides dopamine neurons prepared by the above-described method.

Further, the present invention provides a cellular therapeutic agent for treating Parkinson's disease, including the above-described dopamine neurons.

### BRIEF DESCRIPTION OF THE DRAWINGS

These and/or other aspects will become apparent and more readily appreciated from the following description of the embodiments, taken in conjunction with the accompanying drawings in which:

FIG. 1A illustrates a structure of plasmid DNA expressing mRNA of a dopamine neuron-inducing transcription factor, according to the present invention;

FIG. 1B illustrates a process by which the plasmid DNA expressing mRNA of a dopamine neuron-inducing transcription factor is expressed into mRNA via in vitro transcription;

FIG. 2A illustrates an experimental method for verifying whether the protein of mRNA transfected into HEK293 cells or rat neural precursor cells (rat NPCs) was expressed);

FIG. 2B illustrates immunocytochemistry results of identifying protein expression levels of mRNA of Nurr1 or FoxA2, which is a dopamine neuron-inducing transcription factor, after the HEK293 cells or the rat neural precursor cells (rat NPCs) were each independently transfected therewith, and results showing a cAMP-induced increase in expression levels;

FIG. 3A illustrates an experimental method for verifying whether or not protein expression induced by mRNA transfected into cells is maintained;

FIG. 3B illustrates results showing decreases in protein expression levels for a differentiation period (diff. 0 day, 1 day, 2 days, and 3 days) after rat neural precursor cells (rat NPCs) were transfected with Nurr1 mRNA;

FIG. 4A illustrates an experimental method for verifying whether the rat NPCs are differentiated into dopamine neurons according to repetitive transfection of mRNA into cells;

FIG. 4B illustrates identification results of differentiation into dopamine neurons on differentiation day 3 (diff. 3 days) and on differentiation day 7 (diff. 7 days) while rat neural precursor cells (rat NPCs) were repeatedly transfected with Nurr1 mRNA (N mRNA), and results showing a significant increase in differentiation efficiency when FoxA2 was further expressed;

FIG. 5A illustrates an experimental method for verifying an effect of repetitive transfection of mRNA into cells;

FIG. 5B illustrates results showing that the apoptosis of dopamine neurons was induced from differentiation day 7 (diff. 7 days) as a result of repeatedly transfecting Nurr1 mRNA (N mRNA) into rat neural precursor cells (rat NPCs);

FIG. 6A illustrates an experimental method for verifying the differentiation efficiency of dopamine neurons according to repetitive mRNA transfection under time-based control;

FIG. 6B illustrates results of confirming protein expression maintenance and an increase in differentiation efficiency of dopamine neurons as a result of observing differentiation periods (diff. 10 days, 16 days, 22 days, and 28 days) from differentiation day 7 while rat neural precursor cells (rat NPCs) were repeatedly transfected with Nurr1 mRNA and FoxA2 mRNA;

FIG. 7A illustrates an experimental method for verifying the characteristics and functionality of dopamine neurons differentiated using the method of FIG. 6A;

FIGS. 7B to 7E illustrate results of confirming the expression of dopamine neuron-specific markers (TH, AADC, DAT, VMAT2, and Lmx1A) (see FIGS. 7B and 7C), a dopamine releasing ability (see FIG. 7D), and active sodium current and action potential firing (see FIG. 7E); and

FIG. 8 illustrates results showing chromosomal stability of dopamine neurons differentiated by transfection of Nurr1 mRNA according to the present invention, i.e., the absence of the Nurr1 gene, which was exogenously transfected, as compared to transduction of the Nurr1 gene using a retroviral vector.

### DETAILED DESCRIPTION OF EXEMPLARY EMBODIMENTS

Exemplary embodiments of the present invention will be described in detail below with reference to the accompanying drawings. While the present invention is shown and described in connection with exemplary embodiments thereof, it will be apparent to those skilled in the art that various modifications can be made without departing from the spirit and scope of the present invention.

The inventors of the present invention found that differentiation into mature and functional dopamine neurons having chromosomal stability was enabled by transfecting neural stem cells with mRNA of a dopamine neuron-inducing transcription factor, thus completing the present invention.

Therefore, the present invention provides a method of differentiating neural stem cells or neural precursor cells into dopamine neurons, which includes transfecting neural stem cells or neural precursor cells with mRNA of a dopamine neuron-inducing transcription factor, and dopamine neurons prepared by the method.

The term “differentiation” as used herein refers to a process by which stem cells in an undifferentiated state at an early stage attain characteristics of respective tissues, and for the purpose of the present invention, indicates that neural stem cells or neural precursor cells have characteristics as dopamine neurons.

The term “neural stem cells” as used herein refers to cells having the capacity to differentiate into neurons and glial cells, and these cells may perform cell division 40 times or more, more preferably 50 times or more, and most preferably unlimitedly. The neural stem cells may be obtained from various sources, preferably mammals such as humans, mice, or rats, and more preferably, may be derived from embryos, adult tissues, fetal tissues, or embryonic stem cells (ESCs).

In addition, the term “neural precursor cells” as used herein refer to cells capable of producing offspring which are mature neurons, and the cells may be differentiated from ESCs, adult stem cells, or neural stem cells for use, or may be directly isolated from the ventral midbrain, cerebral cortex, or lateral ganglionic eminence (striatal anlage) of mammals for use.

The term “dopamine neurons” as used herein refers to neurons expressing tyrosine hydroxylase (TH). Dopamine neurons are located specifically in the midbrain substantia nigra, and stimulate the striatum, the limbic system, and the neocortex in vivo to regulate postural reflexes, motions, and compensatory behaviors. In particular, cells should exhibit characteristics of the midbrain to actually function as dopamine neurons in vivo.

In the present invention, the dopamine neuron-inducing transcription factor may be nuclear receptor related 1 (Nurr1) or Forkhead box protein A2 (FoxA2).

Nucleic acids encoding Nurr1 or FoxA2 according to the present invention may be used without limitation as long as they have base sequences encoding Nurr1 or FoxA2, known in the art.

Nurr1 is a transcription factor belonging to steroid/thyroid hormone receptors expressed in midbrain dopamine neurons, and is considered to be expressed in dopamine neurons and play a role in generating midbrain dopamine neurons. The mutation of Nurr1 is known to be associated with dopamine dysfunction-related disorders including Parkinson's disease, schizophrenia, and bipolar disorder, and the expression dysregulation of the Nurr1 gene is known to be associated with rheumatoid arthritis.

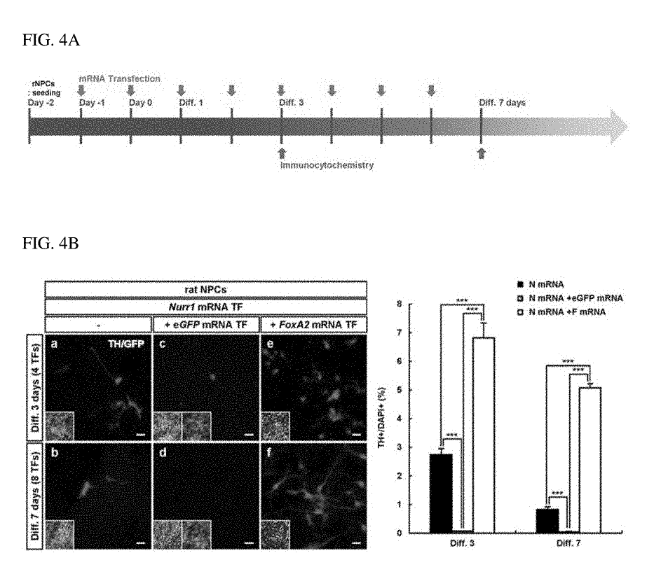

FoxA2 is a transcription factor expressed in the central nervous system, and is known as a transcription factor that plays an essential role in developing and maintaining dopamine neurons. In an embodiment of the present invention, it was confirmed that, when Nurr1 mRNA was transfected into neural precursor cells along with FoxA2 mRNA, the efficiency of differentiation into dopamine neurons was significantly increased, as compared to the case of single transfection of Nurr1 mRNA (see FIG. 4B).

In the present invention, the transfection of mRNA refers to a method of synthesizing a gene to be expressed into messenger RNA and delivering the synthetic mRNA (mRNA transfection). This method enables response to innate antiviral responses when cells are transfected with mRNA including modified 5-methylcytidin and pseudouridine as well as CTP and UTP, which are existing base components, and such a gene expression method through mRNA transfection is simple, has no risk of genetic modification, and has excellent conversion efficiency by high gene control.

The mRNA may be synthesized and prepared by in vitro transcription of plasmid DNA constructed to express mRNA.

In the present invention, the method may further include inducing differentiation of neural stem cells or neural precursor cells into neurons for about 5 days to about 10 days before the transfection of mRNA of Nurr1 and/or FoxA2, which is a dopamine neuron-inducing transcription factor, and differentiation into dopamine neurons may be successfully induced by repeatedly transfecting the mRNA into neural stem cells or neural precursor cells at intervals of about 1 day to about 2 days, preferably 1 day.

In the present invention, the transfection of the mRNA into cells may be performed by those of ordinary skill in the art using a intracellular gene transfection technique known in the art, appropriately selected from among DNA-calcium precipitation, methods using liposomes, methods using polyamines, electroporation, methods using retroviruses, methods using adenoviruses, and the like, and a liposome-mediated method is preferably used in the present invention.

The inventors of the present invention experimentally confirmed the efficiency of differentiation into dopamine neurons through the mRNA transfection and functionality of the differentiated dopamine neurons, through examples.

In one embodiment of the present invention, as a result of transfecting HEK293 cells and neural stem cells with Nurr1 mRNA or FoxA2 mRNA respectively, protein expression was confirmed (see Example 2). In addition, as a result of examining whether the protein expression was maintained, a decrease in protein level was observed within 1 day to 2 days after differentiation, and dopamine neurons produced by differentiation day 7 by using a method of repeatedly transfecting the mRNA were confirmed (see Example 3).

In another embodiment of the present invention, as a result of performing differentiation using the above-described method, induction of the apoptosis of dopamine neurons was observed after 7 days, and to address this, time control-based mRNA transfection was used. More particularly, as a result of repeatedly transfecting cells with mRNA of a dopamine neuron-inducing transcription factor 7 days after neural stem cells started to differentiate into dopamine neurons, it was confirmed that the duration of protein expression maintenance of the transcription factor and the number of dopamine neurons were increased (see Example 4).

In another embodiment of the present invention, as a result of examining the characteristics and functionality of dopamine neurons prepared using the differentiation method of the present invention, the expression of dopamine neuron-specific markers, and dopamine release and electrophysiological characteristics of dopamine neurons were confirmed, from which it was confirmed that differentiation into mature and functional dopamine neurons successfully occurred (see Example 5).

In another embodiment of the present invention, as a result of inducing differentiation into dopamine neurons through the mRNA transfection method of the present invention and a conventional gene transduction method using a retroviral vector, it was confirmed that unlike the retroviral vector, in the case of mRNA transfection, chromosomal stability of the neurons was maintained (see Example 6).

From the above-described examples, it can be confirmed that mature and functional dopamine neurons exhibiting maintained chromosomal stability may be prepared according to the differentiation method of the present invention.

According to another embodiment of the present invention, there is provided a cellular therapeutic agent for treating Parkinson's disease, which includes the above-described dopamine neurons.

The term “cellular therapeutic agent” as used herein refers to a drug used for the purpose of treatment, diagnosis, and prevention, which contains cells or tissues prepared by isolation from a human, culturing, and specific manipulation (US FDA regulations), and refers to a pharmaceutical product used for the purpose of treatment, diagnosis, and prevention, obtained through a series of actions, including growing and screening living autologous, allogenic, or xenogenic cells in vitro in order to restore the function of the cells or tissues or changing the biological characteristics of cells by other methods. The cellular therapeutic agent is broadly classified into a somatic cell therapeutic agent and a stem cell therapeutic agent according to the degree of differentiation of cells.

Hereinafter, exemplary examples will be described to aid in understanding of the present invention. However, the following examples are provided only to more easily understand the present invention and are not intended to limit the scope of the present invention.

### EXAMPLES

### Example 1. Experimental Preparation and Experimental Methods

1-1. Isolation of Rat Neural Precursor Cells (Rat NPCs)

Experimental animals were raised and treated according to the Institutional Animal Care and Use Committee (IACUC, 2016-0194A) guidelines of Hanyang University. Rat neural precursor cells (NPCs) were obtained from the cortex of Sprague-Dawley (SD) rat embryos at an embryonic age of 14.5 days (DaeHan BioLink). Subsequently, rat NPCs isolated from the rat cortex tissues were cultured on a culture dish coated with 15 mg/mL poly-L-ornithine (PLO, Sigma-Aldrich) and 1 mg/mL fibronectin (FN, Sigma-Aldrich) at 37° C. and 5% CO₂.

Next, the rat NPCs were allowed to proliferate in a N₂ medium supplemented with 20 ng/mL of a basic fibroblast growth factor (bFGF, R&D Systems), and to differentiate in a N₂ medium supplemented with 0.2 mM ascorbic acid (Sigma-Aldrich), 20 ng/mL of a brain-derived neurotrophic factor (BDNF, R&D Systems), 20 ng/mL of a glial cell line-derived neurotrophic factor (GDNF, R&D Systems), and 250 μg/mL of dibutyryl-cAMP (db-cAMP, Sigma-Aldrich). Before transfection of the synthetic mRNA, the rat NPCs were treated with each of cAMP derivatives, such as db-cAMP, 10 mM Forskolin (Sigma-Aldrich), and 5 mM NKH477 (Sigma-Aldrich). Thereafter, the synthetic mRNA was transfected into the cells, and then 200 ng/mL of B18R (interferon-gamma inhibitor, eBioscience) was added to the rat NPCs.

1-2. Plasmid Constructs

pcDNA/UTR55A, which is a vector for mRNA synthesis, was constructed using plasmid pcDNA3.1(+) (Invitrogen). More particularly, some restriction enzyme sites (bp 896-930, bp 980-992) of pcDNA3.1+ were replaced by restriction enzymes NheI, BamHI, NotI, and XbaI, and synthesized 5′UTR, 5′UTR reverse, 3′UTR, 3 ′UTR reverse, 55 pA, and 55 pA reverse oligomers (IDT) were annealed and inserted behind the T7 promoter of pcDNA3.1(+). In addition, eGFP, FLAG-tagged Nurr1, and HA-tagged FoxA2 were inserted between the 5′UTR and 3′UTR of the pcDNA/UTR55A.

1-3. mRNA Synthesis

The eGFP/UTR55A, Nurr1(FLAG)/UTR55A, and FoxA2(HA)/UTR55A constructs were linearized by restriction enzyme EcoRV (Takara Bio), and then were used as a template to synthetic mRNA using a MEGAscript T7 Kit (Ambion). Subsequently, the transcription mixture was incubated in vitro at 37° C. for 2 hours. Modified mRNA was generated by adding 5′-methylcytidine and pseudouridine, which are modified ribonucleotides (Trilink Biotechnologies). Then, the modified mRNA was treated with DNase at 37° C. for 15 minutes, and 5′ capping and poly A tailing were performed by adding the ScriptCap m7G Capping System, 2′-O-methyltransferase, and poly(A) polymerase (Epicenter, currently available from CELL® SCRIPT) to the synthetic mRNA. Lastly, the mRNA was precipitated by 2.5 M ammonium acetate (Ambion) and dissolved in a RNA storage solution (Ambion), and then cryopreserved at −70° C.

1-4. mRNA Transfection

One day before transfection, rat cerebral cortex-derived NPCs (50,000 cells/Ø12 mm) were seeded onto a slide glass in a PLO/FN-coated 24-well plate with antibiotic-free growth media. Subsequently, the synthetic mRNAs and the transfection reagent Lipofectamine 2000(Invitrogen) were separately diluted in OPti-MEM media (Invitrogen) and maintained at room temperature for 5 minutes, and then the two solutions were mixed and incubated at room temperature for 20 minutes. The incubated mixture was used to treat the rat NPCs and after 3 hours, the mixture was replaced with growth media or differentiation media to which the recombinant protein B18R was added.

1-5. RT-PCR and Real-Time PCR

To synthesize cDNA, RNAs were extracted from rat NPCs using TRI REAGENT (Molecular Research Center), and then 5 μg of the extracted total RNA was used to synthesize cDNA using the Superscript Kit (Invitrogen). Subsequently, the synthesized cDNA was used for amplification, and the identity of amplicons was confirmed by 1.5% agarose gel electrophoresis.

Real-time PCR analysis was conducted using a conventional method, and was performed in a CFX96 real-time system using iQ SYBR Green Supermix (Bio-Rad), and conditions of the real-time PCR were set as follows: annealing temperature of 60° C. and repetition of 45 cycles. Primer sequences used in the RT-PCR and Real-time PCR are shown in Tables 1 and 2, respectively.

TABLE 1

| | | | SEQ ID |
|---|---|---|---|
| Gene | Direction | Sequence (5′-3′) | NO. |
| eGFP | Forward | ACCCTCGTGACCACCCTGACCT | 1 |
| | Reverse | ACCATGTGATCGCGCTTCTCGT | 2 |
| Nurr1 | Forward | TTCTCCTTTAAGCAATCGCCC | 3 |
| | Reverse | AAGCCTTTGCAGCCCTCACAG | 4 |
| FoxA2 | Forward | GCTCCCTACGCCAATATCAA | 5 |
| | Reverse | CCGGTAGAAAGGGAAGAGGT | 6 |
| TH | Forward | AGCCCCCACCTGGAGTATTTTG | 7 |
| | Reverse | AGCAATCTCTTCCGCTGTGTATTC | 8 |
| VMAT2 | Forward | GCACACAAAATGGGAAGGTGGC | 9 |
| | Reverse | CATTTTTTCCTCCTTAGCAGGTGG | 10 |
| Actin | Forward | TGAGAGGGAAATCGTGCGTG | 11 |
| | Reverse | GTTGGCATAGAGGTCTTTACGG | 12 |

TABLE 2

| | | | SEQ ID |
|---|---|---|---|
| Gene | Direction | Sequence (5′-3′) | NO. |
| TH | Forward | AGCCCCCACCTGGAGTATTTTG | 7 |
| | Reverse | AGCAATCTCTTCCGCTGTGTATTC | 8 |
| AADC | Forward | GCCTTTATCTGTCCTGAGTTCCG | 13 |
| | Reverse | TGATGAGTCCTGAGTCCTGGTGAC | 14 |
| DAT | Forward | GCTGGCACATCTATCCTCTTTGG | 15 |
| | Reverse | CAATGCCCACGACCACATAC | 16 |
| VMAT2 | Forward | GCACACAAAATGGGAAGGTGGC | 9 |
| | Reverse | CATTTTTTCCTCCTTAGCAGGTGG | 10 |
| Lmx1A | Forward | CAGCCTCAGACTCAGGCAAAAGTG | 17 |
| | Reverse | GAACCACACCTGAACCACAC | 18 |
| Actin | Forward | TGAGAGGGAAATCGTGCGTG | 11 |
| | Reverse | GTTGGCATAGAGGTCTTTACGG | 12 |

1-6. Immunocytochemistry

Cultured cells were fixed with 4% formaldehyde (Sigma-Aldrich), and then the fixed cells were treated with 0.1% BSA/PBS, 10% normal goat serum (NGS, Pel-Freez), and 0.03% Triton X-100 (Sigma-Aldrich) for 1 hour. Subsequently, the resulting cells were treated with primary antibodies to allow a reaction to occur at 4° C. overnight, and then treated with biotin-conjugated secondary antibodies (Vector Laboratories) or fluorescence-labeled (DTAF, Rhodamin or Cy3) secondary antibodies (Jackson ImmunoResearch Laboratories). Thereafter, the cells were mounted onto glass slides by VECTASHIELD with a DAPI (Vector Laboratories) mounting medium, and the stained cells were visualized using an epifluorescence microscope (Leica Microsystems) or a confocal microscope (Leica Microsystems). In addition, the lengths of fibers of tyrosine hydroxylase-express sing cells (TH+) were measured using the Leica Application Suite (LAS) image analysis package.

1-7. Production of Recombinant Retrovirus

The inventors of the present invention used a retroviral vector pCL disclosed in a previous study. A control or a gene-expressing retroviral plasmid (retroviral construct) was transfected into 293GPG packaging cells using Lipofectamine 2000. After 72 hours, the virus-containing supernatant was collected every day for 10 days, and 2 μg/mL of polybrene (hexadimethrine bromide, Sigma-Aldrich) was added to the virus supernatant, followed by storage at −70° C.

1-8. DA Release Assay

Dopamine (DA) release assay was performed using the Dopamine Research ELISA Kit (Labor Diagnostika Nord) according to the manufacturer's instructions. At this time, DA-released supernatants were collected under the following two conditions: incubated for 24 hours or stimulated with 56 mM KCl for 30 minutes. DA levels were calculated based on a standard curve generated with a standard control.

1-9. Electrophysiological Analysis

Whole-cell patch clamp recordings from rat NPCs were performed at room temperature (22±1° C.) using an EPC 10 USB amplifier (HEKA Elektronik). The resistances of the pipettes were 4 MΩ to 8 MΩ when filled with a solution consisting of 140 mM K-gluconate, 5 mM di-tris-phosphocreatine, 5 mM NaCl, 4 mM MgATP, 0.4 mM Na₂GTP, 15 mM HEPES, and 2.5 mM Na-pyruvate, and the pH of the solution was adjusted to 7.3 with KOH. Series resistance was compensated by 70% to 80%, current was low-pass-filtered at 2 kHz and sampled at 10 kHz, and a potential of −60 mV was shown. The bath solution included 124 mM NaCl, 26 mM NaHCO₃, 3.2 mM KCl, 2.5 mM CaCl₂, 1.3 mM MgCl₂, 1.25 mM NaHPO₄, and 10 mM glucose, which were saturated with 95% O₂ and 5% CO₂.

1-10. Cell Counting and Statistical Analysis

Cell counting was performed on 10 to 15 randomly selected areas of each of three wells per experimental condition, using a microscope. Each experiment was independently performed at least three times.

All experimental results were expressed as mean±SE, and the paired t test was used for statistical analysis of data.

### Example 2. Verification of Protein Expression by Intracellular Transfection of Synthetic mRNA of Dopamine Neuron-Inducing Transcription Factor

To differentiate the rat cerebral cortex-derived NPCs isolated using the method of Example 1-1 into dopamine neurons, the inventors of the present invention intended to synthesize mRNA of a dopamine neuron-inducing transcription factor as a means for protein expression of the transcription factor and transfect the synthetic mRNA into the rat NPCs.

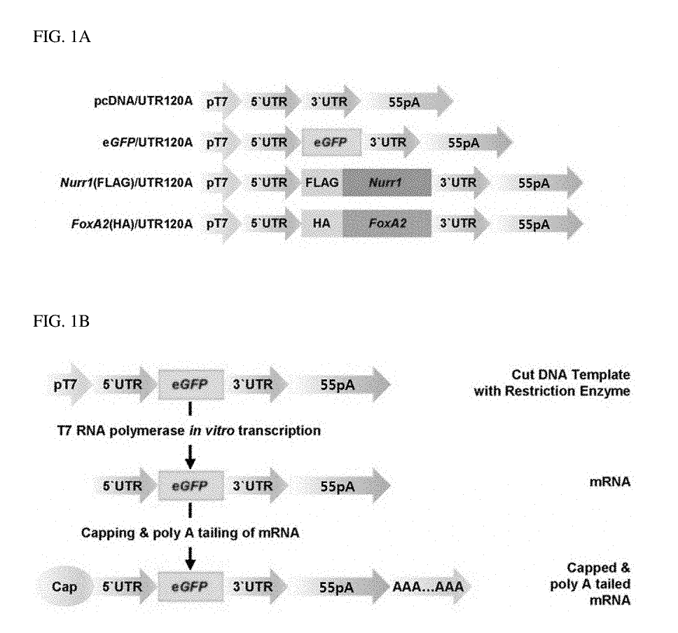

For this, first, to synthesize mRNA that stably expresses a dopamine neuron-inducing transcription factor, plasmid DNAs having a structure illustrated in FIG. 1A were constructed. In particular, each plasmid DNA included a T7 promoter (pT7) for in vitro transcription, UTR (5′UTR and 3′UTR) and a poly A tail (55 pA) for the stability of mRNA, and genes to be expressed (control: eGFP, dopamine neuron-inducing transcription factor: Nurr1 or FoxA2). The plasmid DNAs having the above-described structure have a linear shape by cleavage through restriction enzymes as illustrated in FIG. 1B (Cut DNA template with restriction enzyme), and are synthesized into stable mRNAs through in vitro transcription, capping, and poly A tailing.

To verify whether the mRNAs synthesized through the above process were actually expressed as proteins in cells, the inventors of the present invention transfected HEK293 cells and rat NPCs (or rNPCs), having high intracellular transfection efficiency, with each of the synthetic mRNAs according to the method of Example 1-4 and observed an expression level of each protein according to immunocytochemistry of Example 1-6.

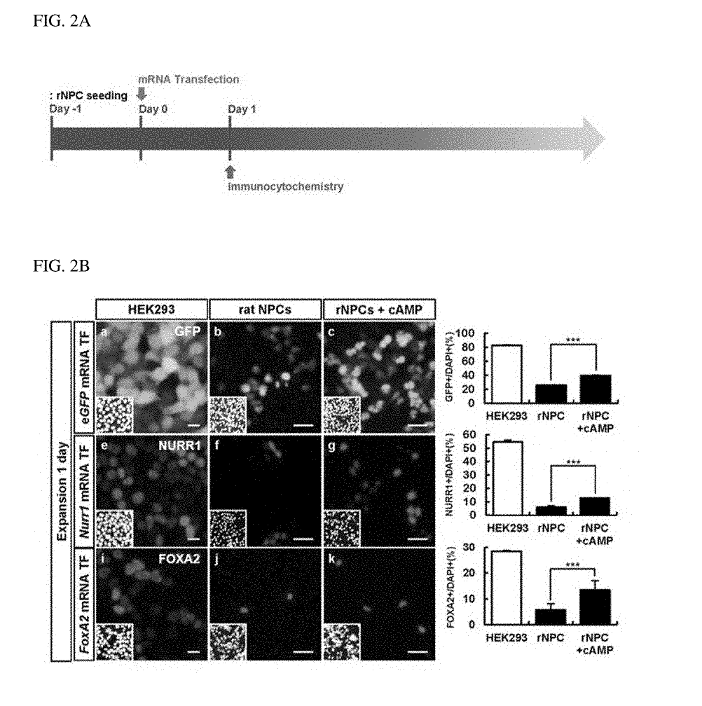

As a result of an experiment performed according to the process of FIG. 2A, as illustrated in FIG. 2B, protein expression of Nurr1 and FoxA2, which are dopamine neuron-inducing transcription factors, by only single mRNA transfection was observed. In addition, as a result of adding cyclic AMP (cAMP) thereto to increase the stability and half-life of mRNAs in cells, a significant increase in protein expression levels was confirmed.

### Example 3. Verification of Protein Expression Maintenance in Cells According to Synthetic mRNA Transfection

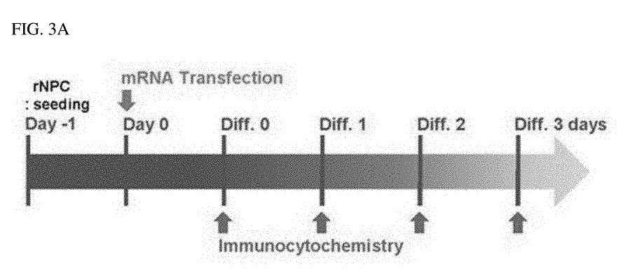

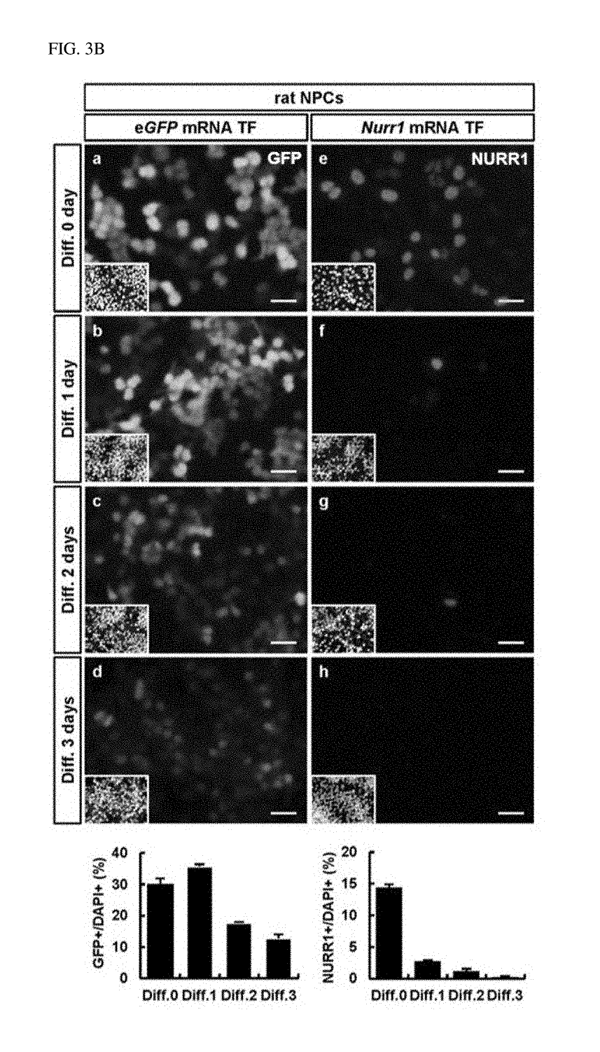

In addition to the results of Example 2, to verify whether protein expression was maintained by intracellularly transfected mRNA, mRNA of Nurr1, which is a dopamine neuron-inducing transcription factor, was transfected into rat NPCs in the same manner as in Example 2, and then protein expression levels were observed for a differentiation period of 1 day to 3 days (diff. 1 to diff. 3) as illustrated in FIG. 3A.

As a result, as illustrated in FIG. 3B, it was confirmed that, when observed until differentiation day 3 (diff. 3), the expression of the Nurr1 protein, which is a dopamine neuron-inducing transcription factor, was maintained only for about 1 day to about 2 days due to rapid degradation of mRNA in cells.

To address this problem, as illustrated in FIG. 4A, the inventors of the present invention repeatedly transfected rNPCs with Nurr1 mRNA at intervals of 1 day so that protein expression was maintained in the cells, and then observed whether the cells were differentiated into dopamine neurons through immunocytochemistry on differentiation day 3 (diff. 3) and differentiation day 7 (diff. 7).

As illustrated in FIG. 4B, as a result of repetitive Nurr1 mRNA transfection into cells (N mRNA TF), differentiation into dopamine neurons expressing tyrosine hydroxylase (TH), which is a dopamine neuron-specific marker, was confirmed, and it was also confirmed that, when the coactivator FoxA2 mRNA was additionally transfected into the cells by using a retrovirus (N mRNA+F mRNA), differentiation into dopamine neurons was significantly increased.

### Example 4. Repetitive Transfection of Synthetic mRNA into Cells Under Time-Based Control and Verification of Effect Thereof

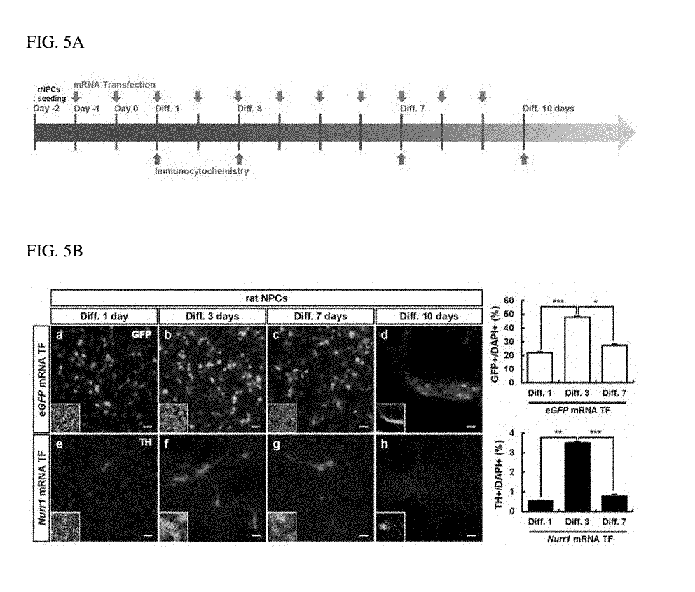

To verify whether repetitive transfection of mRNA of a dopamine neuron-inducing transcription factor into rat NPCs as in Example 3 affected the cells, as illustrated in FIG. 5A, the inventors of the present invention performed observation until differentiation day 10 (diff. 10) while Nurr1 mRNA was repeatedly transfected.

As a result, as illustrated in FIG. 5B, it was confirmed that the apoptosis of dopamine neurons was induced by repetitive mRNA transfection after 7 days of differentiation into dopamine neurons and after 9 days to 10 days, almost no cells were observed.

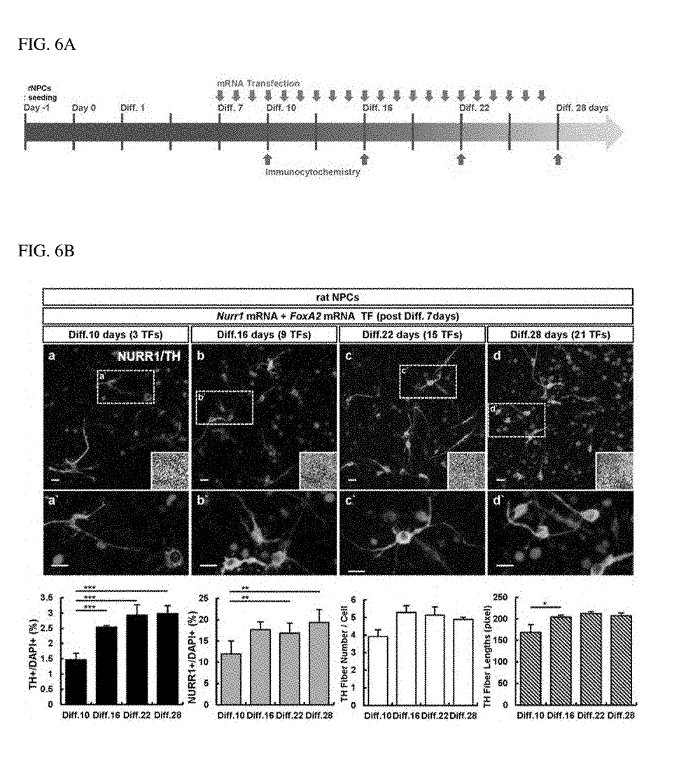

To address these problems, the inventors of the present invention confirmed that the expression of Nurr1 had a delayed expression pattern in the midbrain in which dopamine neurons are present, and tried delayed expression for time-based control of Nurr1 and FoxA2, which are dopamine neuron-inducing transcription factors. More particularly, as illustrated in FIG. 6A, mRNA of a dopamine neuron-inducing transcription factor was repeatedly transfected into rNPCs from day 7 after the rNPCs started to differentiate into dopamine neurons, and the degree of differentiation into dopamine neurons was observed on differentiation day 10 (diff. 10), differentiation day 16 (diff. 16), differentiation day 22 (diff. 22), and differentiation day 28 (diff. 28).

As a result, as illustrated in FIG. 6B, it was confirmed that a maintenance period of the protein expression was further increased through measurement of an expression level of the Nurr1 protein, and by measuring the number of TH-expressing cells (TH+) and the number and length of TH fibers, it was confirmed that the number of mature dopamine neurons was increased.

### Example 5. Verification of Characteristics and Functionality of Dopamine Neurons Differentiated by Transfection of mRNA of Dopamine Neuron-Inducing Transcription Factor

Based on the results of Example 4, the inventors of the present invention intended to observe the characteristics and functionality of dopamine neurons prepared by transfecting mRNA of a dopamine neuron-inducing transcription factor into neural stem cells isolated from the cerebral cortex of rat embryos under time-based control.

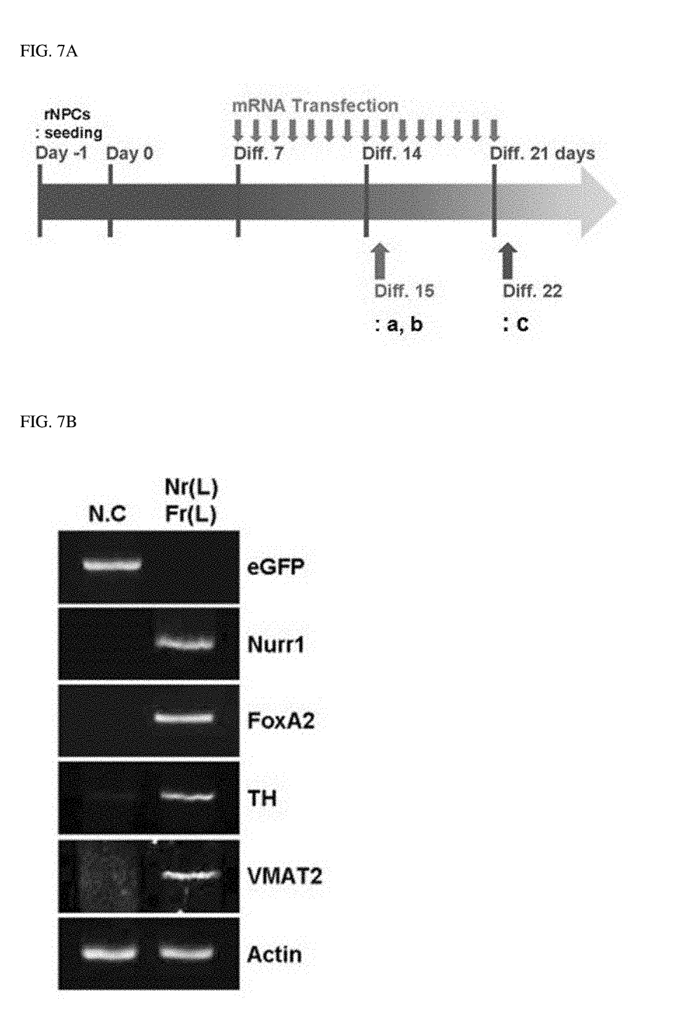

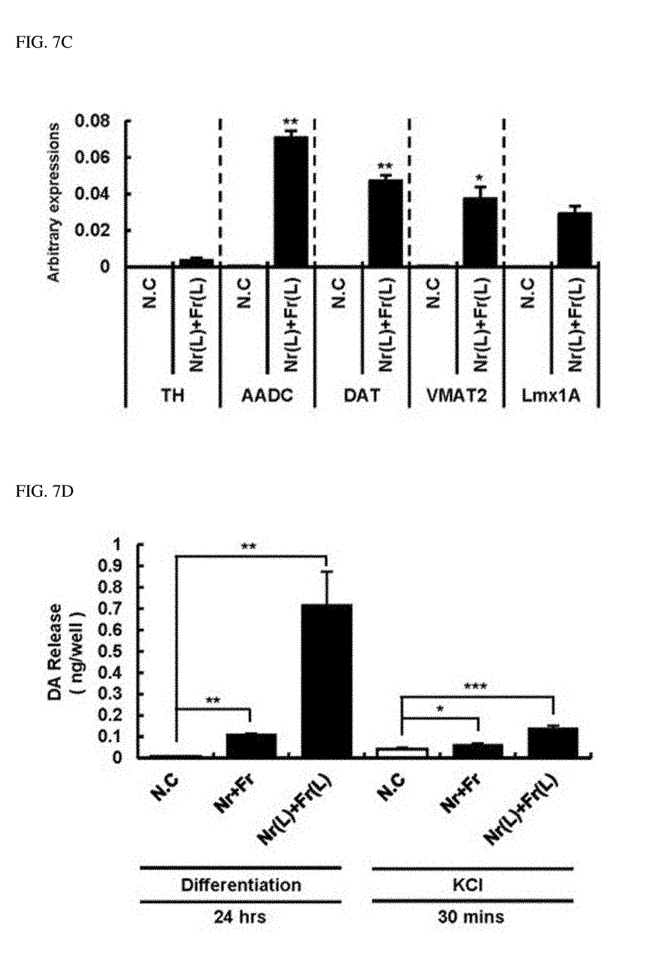

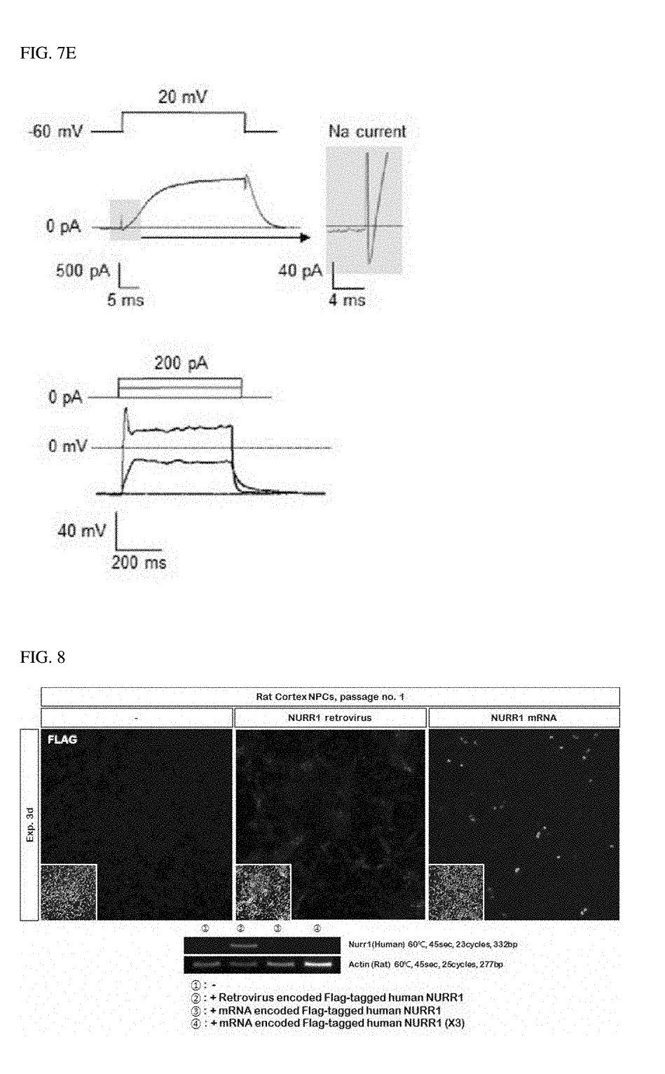

For this, an experiment was carried out according to a process illustrated in FIG. 7A, and then first, the expression of dopamine neuron markers (TH, AADC, DAT, VMAT2, and Lmx1A) was observed by RT-PCR and quantitative real-time PCR on differentiation day 14 (diff. 14). As a result, as illustrated in FIGS. 7B and 7C, it was confirmed that the dopamine neurons obtained through differentiation (Nr(L)Fr(L)) expressed all the markers, as compared to control cells (N.C).

Next, to confirm a dopamine releasing ability of the differentiated dopamine neurons, on differentiation day 15, early dopamine neurons (Nr+Fr) and post dopamine neurons (Nr(L)+Fr(L)) were incubated for 24 hours or stimulated with 56 mM KCl for 30 minutes, and then supernatants were collected and dopamine levels were measured. As a result, as illustrated in FIG. 7D, the amount of released dopamine was significantly the largest in Nr(L)+Fr(L), and a significant difference was shown in the case of incubation for 24 hours as compared to the case of stimulation with KCl. In addition, as a result of performing an electrophysiological experiment on differentiation day 22, as illustrated in FIG. 7E, active sodium current and action potential firing were confirmed.

From the above results, it was confirmed that the differentiated dopamine neurons were mature cells having both characteristics and functionality thereof.

### Example 6. Verification of Maintenance of Chromosomal Stability of Dopamine Neurons

The inventors of the present invention intended to compare chromosomal stability between dopamine neurons differentiated through mRNA transfection according to the present invention and dopamine neurons differentiated through a conventional different gene transfection method. For this, genes were expressed from dopamine neurons in which differentiation was induced by Nurr1 gene transfection using a retroviral vector (Nurr1 retrovirus) and dopamine neurons in which differentiation was induced by Nurr1 mRNA transfection(Nurr1 mRNA), and 3 days after gene expression, genomic DNAs were extracted and gene residual states were measured.

As a result, as illustrated in FIG. 8, when the retroviral vector was used, the retrovirus was randomly inserted into chromosomes of the neurons and remained therein, thus causing chromosomal modification, whereas when mRNA was transfected, no gene insertion into chromosomes occurred, from which it was confirmed that chromosomal stability was maintained. From these results, it can be seen that unlike existing methods of inducing dopamine neurons using a viral vector, dopamine neurons prepared using mRNA are cells with chromosomal stability, and thus may exhibit excellent effectiveness and safety for clinical application.

As is apparent from the foregoing description, a method of differentiating into dopamine neurons, according to the present invention, can enable preparation of mature and functional dopamine neurons having chromosomal stability by synthesizing a dopamine neuron-inducing transcription factor into a mRNA form, which has no risk of genetic modification, and transfecting the synthetic mRNA, unlike existing methods using retroviral vectors, and thus can be usefully used in the clinical field for the treatment of Parkinson's disease.

The foregoing description of the present invention is provided for illustrative purposes, and it will be understood by those of ordinary skill in the art to which the present invention pertains that the invention may be easily modified in many different forms without departing from the spirit or essential characteristics of the present invention. Therefore, it is to be understood that the above-described embodiments are illustrative in all aspects and not restrictive.

What is claimed is:

1. A method of differentiating neural stem cells or neural precursor cells into dopamine neurons, the method comprising transfecting neural stem cells or neural precursor cells with mRNA of a dopamine neuron-inducing transcription factor.

2. The method of claim 1, wherein the dopamine neuron-inducing transcription factor is nuclear receptor related 1 (Nurr1) or Forkhead box protein A2 (FoxA2).

3. The method of claim 1, further comprising, before the transfecting, inducing differentiation of the neural stem cells or the neural precursor cells into neurons for about 5 days to about 10 days.

4. The method of claim 1, wherein the mRNA is repeatedly transfected into the neural stem cells or the neural precursor cells at an interval of about 1 day to about 2 days.

5. Dopamine neurons prepared using the method of claim 1.

6. A cellular therapeutic agent for treating Parkinson's disease, the cellular therapeutic agent comprising the dopamine neurons of claim 5.


SEQUENCE LISTING (18 sequences)

SEQ ID NO: 1 - DNA, 22 bp, Artificial Sequence
  eGFP_Forward
accctcgtgaccaccctgacct

SEQ ID NO: 2 - DNA, 22 bp, Artificial Sequence
  eGFP_Reverse
accatgtgatcgcgcttctcgt

SEQ ID NO: 3 - DNA, 21 bp, Artificial Sequence
  Nurr1_Forward
ttctcctttaagcaatcgccc

SEQ ID NO: 4 - DNA, 21 bp, Artificial Sequence
  Nurr1_Reverse
aagcctttgcagccctcacag

SEQ ID NO: 5 - DNA, 20 bp, Artificial Sequence
  FoxA2_Forward
gctccctacgccaatatcaa

SEQ ID NO: 6 - DNA, 20 bp, Artificial Sequence
  FoxA2_Reverse
ccggtagaaagggaagaggt

SEQ ID NO: 7 - DNA, 22 bp, Artificial Sequence
  TH_Forward
agcccccacctggagtattttg

SEQ ID NO: 8 - DNA, 24 bp, Artificial Sequence
  TH_Reverse
agcaatctcttccgctgtgtattc

SEQ ID NO: 9 - DNA, 22 bp, Artificial Sequence
  VMAT2_Forward
gcacacaaaatgggaaggtggc

SEQ ID NO: 10 - DNA, 24 bp, Artificial Sequence
  VMAT2_Reverse
cattttttcctccttagcaggtgg

SEQ ID NO: 11 - DNA, 20 bp, Artificial Sequence
  Actin_Forward
tgagagggaaatcgtgcgtg

SEQ ID NO: 12 - DNA, 22 bp, Artificial Sequence
  Actin_Reverse
gttggcatagaggtctttacgg

SEQ ID NO: 13 - DNA, 23 bp, Artificial Sequence
  AADC_Forward
gcctttatctgtcctgagttccg

SEQ ID NO: 14 - DNA, 24 bp, Artificial Sequence
  AADC_Reverse
tgatgagtcctgagtcctggtgac

SEQ ID NO: 15 - DNA, 23 bp, Artificial Sequence
  DAT_Forward
gctggcacatctatcctctttgg

SEQ ID NO: 16 - DNA, 20 bp, Artificial Sequence
  DAT_Reverse
caatgcccacgaccacatac

SEQ ID NO: 17 - DNA, 24 bp, Artificial Sequence
  Lmx1A_Forward
cagcctcagactcaggcaaaagtg

SEQ ID NO: 18 - DNA, 20 bp, Artificial Sequence
  Lmx1A_Reverse
gaaccacacctgaaccacac